(19) United States
(12) Patent Application Publication
(10) Pub. No.: US 2015/0335392 A1
(43) Pub. Date: Nov. 26, 2015

(54) WOUND MAPPING MARKERS

(71) Applicant: Thomas J. Richards, Chino Hills, CA (US)

(72) Inventor: Thomas J. Richards, Chino Hills, CA (US)

(21) Appl. No.: 14/712,825

(22) Filed: May 14, 2015

Related U.S. Application Data
(60) Provisional application No. 62/002,087, filed on May 22, 2014.

(51) Int. Cl. A61B 19/00

(52) U.S. Cl. CPC A61B 19/54; A61B 2019/5404; A61B 2019/5491; A61B 2019/545

(57) ABSTRACT

A wound mapping marker, including a backing sheet, and a flexible marker sticker adhered to the backing sheet. The sticker is configured for removal from the backing sheet and for adherence to the body of a subject adjacent a wound or other body feature. A set of the markers may be disposed on a backing sheet.

### CROSS-REFERENCE TO RELATED APPLICATIONS

This application claims priority from U.S. Provisional Application No. 62/002,087, filed May 22, 2014, the entire contents of which application are hereby incorporated by reference.

### BACKGROUND

Healthcare facilities must document wounds on the patient when admitting into the facility. Law enforcement investigators also document wounds on victims. In both cases, it can be difficult to track and locate the wounds in the medical records when reviewing them at a later date. The more wounds a patient has the harder it is to differentiate, especially if they are relatively close to each other. Currently, investigators will map the wounds on paper in the medical record, however it is still difficult to determine one wound from the other when there is more than one wound on the same body location. Even with a photograph this is difficult. This is compounded when time passes between assessments and different people are reviewing the medical record.

### BRIEF DESCRIPTION OF THE DRAWINGS

Features and advantages of the disclosure will readily be appreciated by persons skilled in the art from the following detailed description when read in conjunction with the drawing wherein:

FIG. 1 is a diagrammatic view of an exemplary embodiment of a wound mapping marker, with a numeric representation and a scale representation. FIG. 1A is a diagrammatic cross-section of the marker of FIG. 1, taken along line 1A-1A of FIG. 1.

FIG. 2 is a diagrammatic view of another exemplary embodiment of a wound mapping marker, with an arrow designation.

FIG. 3 is a diagrammatic view of yet another exemplary embodiment of wound mapping markers, depicting a set of markers on a sheet.

FIG. 4 is a diagrammatic view of a subject's arm, illustrating placement of wound mapping markers in relation to wound sites on the arm.

FIG. 5 is a diagrammatic view of another exemplary embodiment of a mapping marker.

FIG. 6 illustrates another exemplary embodiment of a mapping marker.

### DETAILED DESCRIPTION

In the following detailed description and in the several figures of the drawing, like elements are identified with like reference numerals. The figures may not be to scale, and relative feature sizes may be exaggerated for illustrative purposes.

Exemplary embodiments of wound mapping markers (WMM), when used with photography, allow the healthcare or law enforcement investigator to track wounds in his or her documentation.

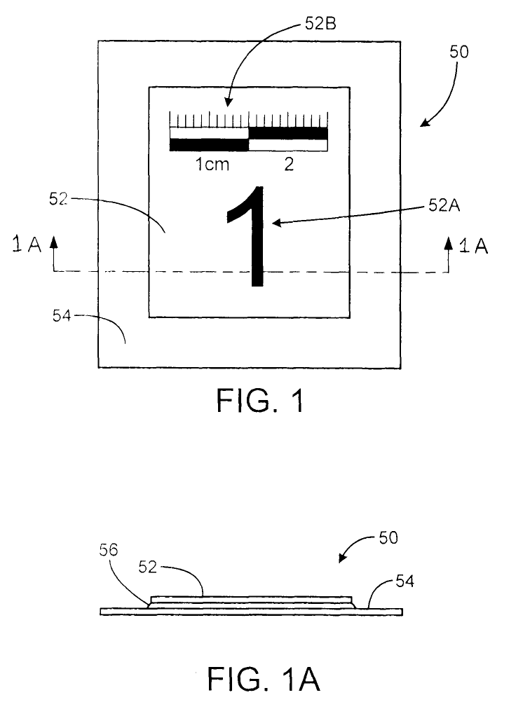

An exemplary embodiment of a WMM is illustrated in FIGS. 1 and 1A, and is a small sticker or marker 52 which has a number or letter indicia 52A and a small scale or ruler indicia 52B formed or imprinted on it. The number or letter is configured to provide identification of a wound, observation or feature on a patient or object, to differentiate where the wound, observation or feature is on the patient or object. The scale is configured to indicate the size of the wound or feature. Each marker is large enough to display a large number or letter formed or printed on it so that it can be readily seen in a photograph. An exemplary size of marker 52 is 2.5 cm wide by 3 cm high, with a font size of 66. The printed numbers will typically be in black ink, but can be printed in different colors. The inks can also be made of different materials or characteristics, such as waterproof inks, inks which glow in the dark, or fluorescent ink or inks which react when illuminated with particular types or wavelengths of light. The sticker may have a white background on which the ink is printed. This background can also be made with different colors, or the marker 52 can be fabricated of a clear or translucent material.

The stickers 52 may be made of paper, plastic, vinyl, etc., and configured to removably adhere to a backing sheet 54, e.g. through an adhesive layer 56 (FIG. 1A) or other adhesion means, or to the subject's body or object while the wound or object is being photographed. The adhesive layer, or a portion thereof, may release from the backing sheet when the sticker is removed, to adhere to the object or subject's body during image capture. The sticker is readily removable from the backing sheet, and subsequently from the subject's body or other object.

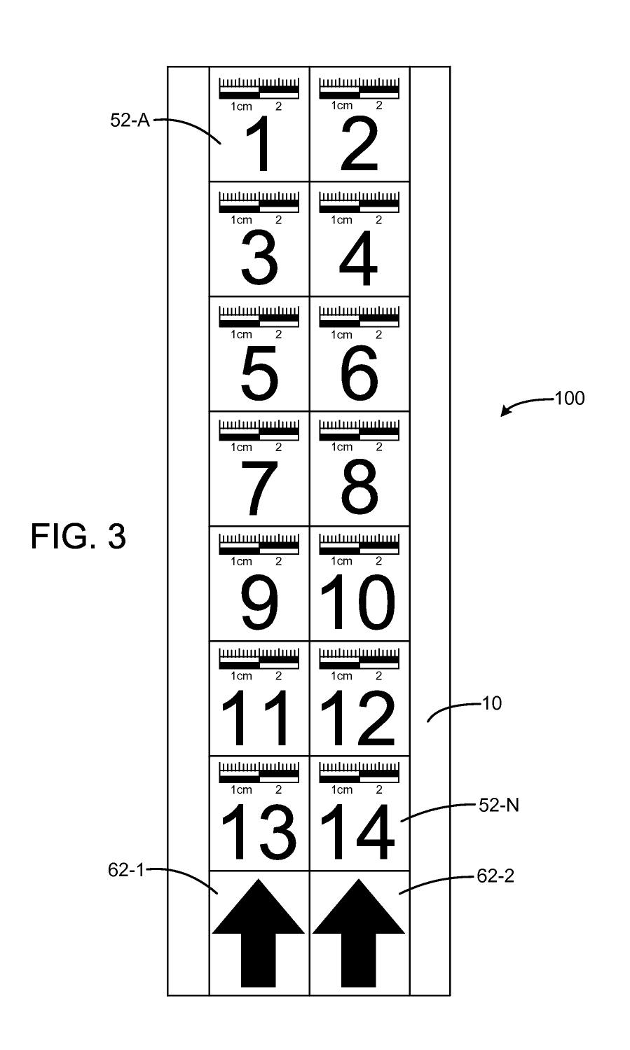

The stickers or markers 52 are preferably thin, e.g., paper thin, flexible layers, on which is formed or printed the number, letter, scale or other indicia. The individual markers can be packaged or laid out on a single sheet with a sequence of numbered individual markers on it, as shown in FIG. 3. The individual markers can also be packaged on many sheets with each sheet containing a set of identical markers, e.g., a sheet of 1's and a sheet of 2's, etc. The individual markers are removably attached relative to the other markers on the sheet to allow selective removal of an individual marker sticker from the backing sheet without disturbing the remaining marker stickers on the backing sheet.

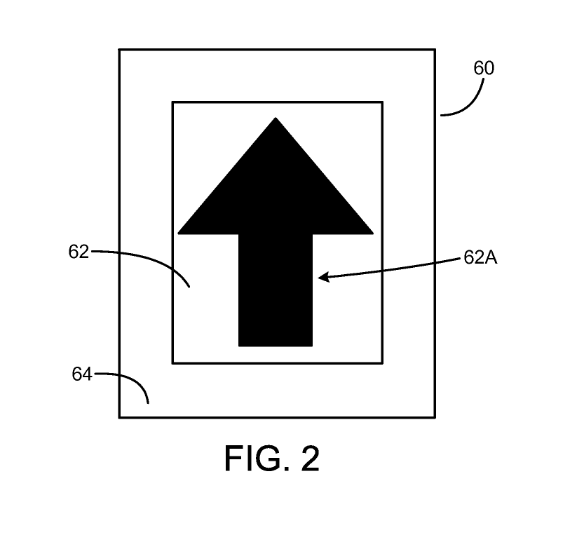

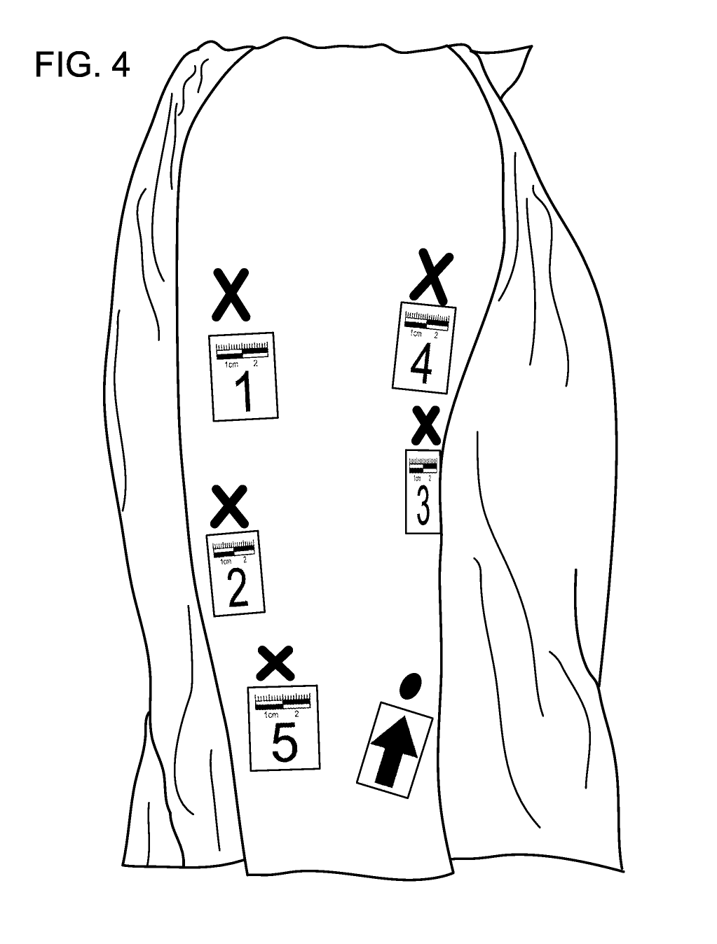

FIG. 2 illustrates another embodiment of a WMM 60, in this case a sticker 62 on a backing sheet 64, with a directional arrow representation 62A. The arrow representation provides an alternate way to highlight or draw attention to an observation on a patient or object that needs to be documented. For example, in FIG. 4, an arrow is used to mark an observation (represented by a dot in FIG. 4) which is different from the other observations (represented by “x”s) on the subject's arm. The WMM with an arrow representation may also be used when numbers are not necessary, and also may be used in conjunction with other scales such as traditional rulers.

The WMMs may be fabricated as individual markers on individual backing sheets, as illustrated in FIGS. 1 and 2, or configured in sets on a common backing sheet. FIG. 3 illustrates a further embodiment, of a WMM set 100, in which individual WMMs 52-A . . . 52-N and 62-A, 62-B are supported on a backing sheet 110, and configured to be removed from the sheet individually. The WMMs 52-A . . . 52-N in this embodiment are markers with numeric indicia, from 1 to 14 in this example, and each of these markers also includes a scale indicia as well. The set 100 in this case also includes arrow markers 62-1 and 62-2. The number of individual markers in the set, as well as the constitution of the individual markers, may vary.

To use an individual WMM such as illustrated in FIGS. 1 and 2, or a WMM set such as set 100 in FIG. 3, in an exemplary application for documenting wounds, the individual marker is removed from the backing (54, 64 or 10) and placed next to a wound. This process is continued until all the wounds are accounted for. There could be one or any number of wounds to mark; one example is illustrated in FIG. 4, in which five wounds will be marked. On an assessment sheet, or form if using electronic medical records, the observations of the wounds may be documented in a legend, wound one through five in the foregoing example. When finished with the mapping of the wounds, a photograph of the wounds is taken, showing the layout of the wounds on the body and with respect to each other. FIG. 4 depicts a simplified representation of an exemplary wound photograph, showing in side profile a subject's arm with several wounds mapped and marked by WMMs. The photograph is then stored either in print form, as part of an electronic record, or both.

The WMM system is particularly useful in documenting wounds on a body. One benefit of mapping the wounds using a WMM is to allow for a better organization of the wound assessment in the medical record. It keeps the information in an order so that when a person reviews the medical record, he or she will know which wound description is related to the actual wound on the patient or victim. When reviewing the photograph, it makes for quick identification of the location of the wound on the body. Otherwise, if there are five wounds on an arm, the reviewer would not know which wound description to apply to the wound in the photograph. The use of larger fonts allows for photographs from a distance for an entire region of the body, and also allows for close-up photography during the same assessment, keeping the wounds organized.

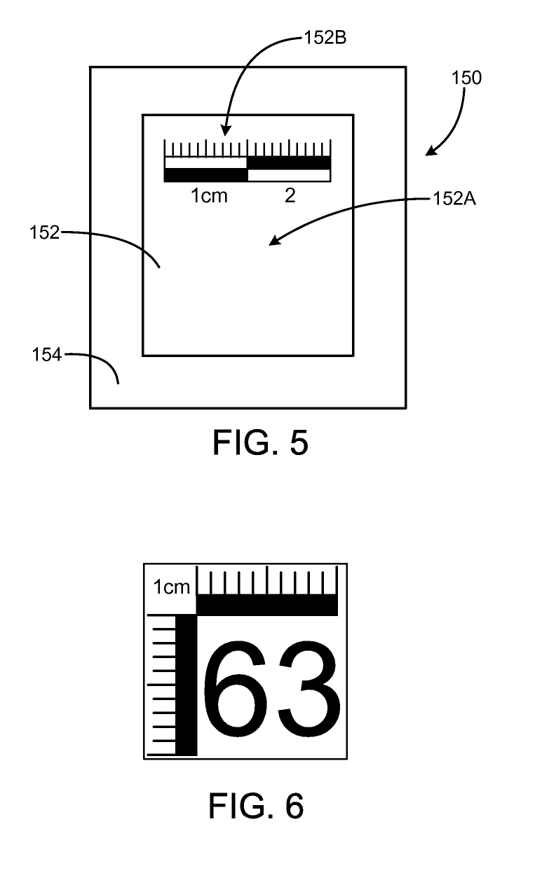

Although the foregoing has been a description and illustration of specific embodiments of the subject matter, various modifications and changes thereto can be made by persons skilled in the art without departing from the scope and spirit of the invention. For example, a wound marker may include a scale, but omit the number or letter, with a blank space on the wound marker for the user to write a number or letter, e. g., using a permanent marker or the like. FIG. 5 shows an embodiment of a wound marker 150, in which the sticker 152 on backing 154 has a blank area 152A, and a scale 152B. The blank area is configured to provide space for the user to hand write an identifying indicia such as a number or letter. In another embodiment illustrated in FIG. 6, the mapping marker may include more than one scale, e.g. scales oriented along transverse sides of the marker sticker. In the example of FIG. 6, the sticker is a square, 1.4 cm by 1.4 cm.

What is claimed is:

1. A mapping marker, comprising:
a backing sheet; and
a flexible marker sticker adhered to the backing sheet and configured for removal from the backing sheet and for adherence to a subject's body adjacent a wound or body feature or to an object, the marker sticker having an indicia comprising a number, letter, scale or directional indicia configured to provide identification of the wound, body feature or object in a photographic image of said wound, body feature or object with the marker sticker.

2. The marker of claim 1, wherein the indicia comprises a number or letter and at least one scale reflecting a length dimension.

3. The marker of claim 1, wherein the indicia comprises a directional arrow indicia.

4. The marker of claim 1, wherein the flexible marker sticker has a size of 2.4 centimeters (cm) by 3 cm.

5. The marker of claim 1, further comprising an adhesive layer for removably adhering the marker sticker to the backing sheet, the adhesive layer configured to release from the backing sheet with the marker sticker to adhere to the subject's body or object while being photographed, and to readily release from the subject's body or object subsequent to said photographing.

6. The marker of claim 5, wherein the marker sticker is fabricated of paper or plastic.

7. The marker of claim 6, wherein the marker sticker is configured to provide a white background for said indicia.

8. A wound mapping marker set, comprising:
a backing sheet; and
a plurality of individual flexible marker stickers adhered to the backing sheet and configured for individual removal from the backing sheet and for adherence to a subject's body adjacent a wound or other body feature, each sticker having an indicia comprising a number, letter, scale or directional indicia configured to provide marking or identification of the wound or other body feature in a photographic image, and wherein each of said markers are removably attached relative to the other markers on the sheet to allow selective removal of an individual marker sticker from the backing sheet without disturbing the remaining marker stickers on the backing sheet.

9. The marker set of claim 8, wherein said marker stickers are identical to each other.

10. The marker set of claim 8, wherein said marker stickers are laid out on said backing sheet to provide marker stickers defining a sequence of numbers or letters.

11. The marker set of claim 10, wherein the marker stickers further comprise a marker sticker in which the indicia includes an arrow and a scale.

12. The marker set of claim 8, wherein the indicia on each of said marker stickers comprises a number or letter and a scale reflecting a length dimension.

13. The marker set of claim 8, wherein the indicia comprises a directional arrow indicia.

14. The marker set of claim 8, wherein each flexible marker sticker has a size of 2.4 centimeters (cm) by 3 cm.

15. The marker set of claim 8, further comprising an adhesive layer for removably adhering each marker sticker to the backing sheet, the adhesive layer configured to release from the backing sheet with the marker sticker to adhere to the subject's body or object while being photographed, and to readily release from the subject's body or object subsequent to said photographing.

16. The marker set of claim 15, wherein each marker sticker is fabricated of paper or plastic.

17. The marker set of claim 16, wherein the marker sticker is configured to provide a white background for said indicia.

18. A mapping marker, comprising:
a backing sheet; and
a flexible marker sticker adhered to the backing sheet and configured for removal from the backing sheet and for adherence to a subject's body adjacent a wound or body feature or to an object, the marker sticker having blank area configured to receive a handwritten indicia comprising a number, letter, or directional indicia to provide identification of the wound, body feature or object in a photographic image of said wound, body feature or object with the marker sticker.

19. The marker of claim 18, wherein the marker sticker comprises a scale reflecting a length dimension.

20. The marker of claim 18, further comprising an adhesive layer for removably adhering the marker sticker to the backing sheet, the adhesive layer configured to release from the backing sheet with the marker sticker to adhere to the subject's body or object while being photographed, and to readily release from the subject's body or object subsequent to said photographing.
